(19) United States
(12) Patent Application Publication
(10) Pub. No.: US 2004/0115668 A1
(43) Pub. Date: Jun. 17, 2004

(54) Human hyperpolarization-activated cyclic nucleotide-gated cation channel hcn3

(76) Inventors: Kimberly A. Folander, Bethlehem, PA (US); Yuan Liu, Lansdale, PA (US); Edward McKenna, Lansdale, PA (US); Richard J Swanson, Lansdale, PA (US)

Correspondence Address: MERCK AND CO INC, P O BOX 2000, RAHWAY, NJ, 070650907

(21) Appl. No.: 10/451,127

(22) PCT Filed: Dec. 14, 2001
(86) PCT No.: PCT/US01/47903

(51) Int. Cl. C12Q 1/68; C07H 21/04; C07K 14/705

(52) U.S. Cl. 435/6; 435/69.1; 435/320.1; 435/325; 530/350; 536/23.5

(57) ABSTRACT

The present invention is directed to novel human DNA sequences encoding human HCN3 proteins, the protein encoded by the DNA sequences, vectors comprising the DNA sequences host cells containing the vectors, and methods of identifying inhibitors and activators of cation channels containing the human HCN3 proteins.

### CROSS-REFERENCE TO RELATED APPLICATIONS

Not applicable.

### STATEMENT REGARDING FEDERALLY-SPONSORED R&D

Not applicable.

### REFERENCE TO MICROFICHE APPENDIX

Not applicable.

### FIELD OF THE INVENTION

The present invention is directed to novel human DNA sequences encoding a hyperpolarization-activated cyclic nucleotide-gated cation channel (HCN3), proteins encoded by the DNA sequences, methods of expressing the proteins in recombinant cells, and methods of identifying activators and inhibitors of HCN3.

The HCN genes encode a family of cation channels that are believed to carry a current known as Iₕ or I_{q} in neural tissue and I_{f} in cardiac tissue. This current is activated by hyperpolarization beyond about −50 to −70 mV, does not inactivate, is carried by both Na⁺ and K⁺, exhibits a small single channel conductance (about 1 pS), and has the effect of slowly depolarizing a cell toward the Iₕ reversal potential of about −30 mV. The voltage dependence of Iₕ can be modulated by cyclic nucleotides such as cAMP or cGMP. The Iₕ current can contribute significantly to the total current at subthreshold membrane potentials, and thus can be an important factor in the regulation of neuronal firing and cardiac contraction.

Three major roles for the Iₕ current have been postulated in neurons: (a) Iₕ contributes to the cell's resting membrane potential; (b) Iₕ can modulate the summation of synaptic inputs into the neuron, e.g., by counteracting hyperpolarizing signals from inhibitory postsynaptic potentials; and (c) Iₕ contributes to the generation of “pacemaker” or oscillatory activity (i.e., rhythmic, spontaneous firing of action potentials).

In the heart, the I_{f} current arises following repolarization of an action potential, which returns the cell to its hyperpolarized resting membrane potential. In pacemaker regions of the heart, such as the sinoatrial node, this hyperpolarization activates I_{f}, which leads to a slow depolarization of the myocyte, eventually returning the membrane potential to the action potential threshold, and triggering another action potential. The larger the I_{f} current, the more rapid the return to the action potential threshold and the faster the heart will beat. Agents that stimulate the heart by stimulating the β-adrenergic receptor act, in part, through the I_{f} current. Such agents lead to an increase in intracellular cAMP which shifts the voltage dependence of the I_{f} current towards more positive (i.e., depolarized) levels, resulting in faster entry of this current into its role in moving the cell back toward the action potential threshold.

For reviews of the Iₕ/I_{f} current, see Clapham, 1998, Neuron 21:5-7; Lüthi & McCormick, 1998, Neuron 21:9-12; Pape, 1996, Ann. Rev. Physiol. 58:299-327; DiFrancesco, 1993, Ann. Rev. Physiol. 55:455-472.

Certain HCN genes and their encoded protein products have been identified. The DNA and deduced amino acid sequences, as well as some electrophysiological properties, of human HCN2 and human HCN4 have been disclosed (Vaccari et al. 1999. Biochim. Biophys. Acta 1446:419-425; Seifert et al., 1999,

Proc. Natl. Acad. Sci. USA 96:9391-9396; Ludwig et al., 1999, EMBO J. 18:2323-2329; GenBank accession nos. AF065164 and AJ012582 (HCN2); GenBank accession nos. AJ132429 and AJ238850 (HCN4)). GenBank accession no. AF064876 represents a partial HCN1 sequence. Certain fragments of human HCN3 have appeared in certain databases (GenBank accession nos. AI571225; AQ625620). Full length mouse HCN1, HCN2, and HCN3 have been cloned as has a partial mouse cDNA encoding HCN4 (Santoro et al., 1998, Cell 93:717-729; Ludwig et al., 1998, Nature 393:587-591). Examination of the cDNAs encoding HCN channels revealed that the HCN proteins represent a family of ion channels having six putative transmembrane domains (S1-S6) and a cAMP binding domain. Functional expression of human HCN2 in a kidney cell line produced currents with properties similar to those of the heart I_{f} current (Vaccari et al., 1999, Biochim. Biophys. Acta 1446:419-425).

It is desirable to discover as wide a variety as possible of novel cation channels, especially those from humans and those exhibiting restricted tissue expression. Such novel cation channels would be attractive targets for drug discovery, useful in counterscreens for a variety of other drug targets, and would be valuable research tools for understanding more about ion channel biology.

### SUMMARY OF THE INVENTION

The present invention is directed to a novel human DNA sequence encoding human HCN3, a hyperpolarization-activated cyclic nucleotide-gated cation channel. The present invention also includes certain polymorphic variants of human HCN3. The present invention includes DNA comprising the nucleotide sequences shown as SEQ.ID.NOs.:1, 3, and 5 as well as DNA comprising the coding regions of SEQ.ID.NOs.:1, 3, and 5. Also provided are proteins encoded by the novel DNA sequences. The human HCN3 proteins of the present invention comprise the amino acid sequences shown as SEQ.ID.NOs.:2, 4, and 6 as well as fragments thereof. Methods of expressing the novel human HCN3 proteins in recombinant systems are provided. Also provided are methods of using human HCN3 as a drug target by identifying activators and inhibitors of cation channels comprising human HCN3 proteins. Also provided are methods of using the novel human HCN3 proteins and DNA encoding these HCN3 proteins in counterscreens for assays designed to identify activators and inhibitors of other drug targets.

### BRIEF DESCRIPTION OF THE DRAWINGS

FIG. 1A shows a cDNA sequence encoding human HCN3 (SEQ.ID.NO.:1) and FIG. 1B shows the corresponding amino acid sequence (SEQ.ID.NO.:2). The start ATG codon in FIG. 1A is at position 9-11; the stop codon is at position 2331-2333.

FIG. 2A shows a cDNA sequence encoding human HCN3 with a single nucleotide polymorphism (SEQ.ID.NO.:3) as compared to (SEQ.ID.NO.:1). Position 1051 in SEQ.ID.NO.:3 is C rather than G as in SEQ.ID.NO.:1. FIG. 2B shows the amino acid sequence (SEQ.ID.NO.:4) encoded by SEQ.ID.NO.:3. SEQ.ID.NO.:4 differs from SEQ.ID.NO.:2 in having an A rather than a G at position 348.

FIG. 3A shows a cDNA sequence encoding human HCN3 with a single nucleotide polymorphism (SEQ.ID.NO.:5) as compared to (SEQ.ID.NO.:1). Position 1795 in SEQ.ID.NO.:5 is T rather than G as in SEQ.ID.NO.:1. FIG. 3B shows the amino acid sequence (SEQ.ID.NO.:6) encoded by SEQ.ID.NO.:5. SEQ.ID.NO.:6 differs from SEQ.ID.NO.:2 in having an L rather than an R at position 596.

FIGS. 4A-B shows an amino acid sequence alignment of human HCN3 (HCN3_Hum; SEQ.ID.NO.:2), mouse HCN3 (HCN3_Mus; SEQ.ID.NO.:7; GenBank accession no. AJ225124), and rat HCN3 (HCN3_Rat; SEQ.ID.NO.:8; GenBank accession no. AF247452). The consensus sequence is SEQ.ID.NO.:20.

FIG. 5 shows the results of a multi-tissue Northern blot demonstrating that human HCN3 is expressed in a variety of tissues, including brain and heart. Lanes: 1=heart; 2=brain; 3=placenta; 4=lung; 5=liver; 6=skeletal muscle; 7=kidney; 8=pancreas; 9=spleen; 10=thymus; 11=prostate; 12=testis; 13=ovary; 14=small intestine; 15=colon; 16=peripheral blood leukocytes.

### DETAILED DESCRIPTION OF THE INVENTION

For the purposes of this invention:

“Substantially free from other proteins” means at least 90%, preferably 95%, more preferably 99%, and even more preferably 99.9%, free of other proteins. Thus, a human HCN3 protein preparation that is substantially free from other proteins will contain, as a percent of its total protein, no more than 10%, preferably no more than 5%, more preferably no more than 1%, and even more preferably no more than 0.1%, of proteins that are not human HCN3 proteins. Whether a given human HCN3 protein preparation is substantially free from other proteins can be determined by conventional techniques of assessing protein purity such as, e.g., sodium dodecyl sulfate polyacrylamide gel electrophoresis (SODS-PAGE) combined with appropriate detection methods, e.g., silver staining or immunoblotting.

“Substantially free from other nucleic acids” means at least 90%, preferably 95%, more preferably 99%, and even more preferably 99.9%, free of other nucleic acids. Thus, a human HCN3 DNA preparation that is substantially free from other nucleic acids will contain, as a percent of its total nucleic acid, no more than 10%, preferably no more than 5%, more preferably no more than 1%, and even more preferably no more than 0.1%, of nucleic acids that are not human HCN3 nucleic acids. Whether a given human HCN3 DNA preparation is substantially free from other nucleic acids can be determined by conventional techniques of assessing nucleic acid purity such as, e.g., agarose gel electrophoresis combined with appropriate staining methods, e.g., ethidium bromide staining.

A “conservative amino acid substitution” refers to the replacement of one amino acid residue by another, chemically similar, amino acid residue. Examples of such conservative substitutions are: substitution of one hydrophobic residue (isoleucine, leucine, valine, or methionine) for another; substitution of one polar residue for another polar residue of the same charge (e.g., arginine for lysine; glutamic acid for aspartic acid); substitution of one aromatic amino acid (tryptophan, tyrosine, or phenylalanine) for another.

A polypeptide has “substantially the same biological activity as human HCN3” if that polypeptide is able to either form a functional cation channel by itself, i.e., as a homomultimer, having properties similar to that of human HCN3 channels, or combine with at least one other cation channel subunit (e.g., HCN1, HCN2, or HCN4) so as to form a complex that constitutes a functional cation channel where the polypeptide confers upon the complex (as compared with the other subunit alone) altered electrophysiological or pharmacological properties that are similar to the electrophysiological or pharmacological properties that the human HCN3 protein having SEQ.ID.NO.:2 confers on the complex and where the polypeptide has an amino acid sequence that is at least about 50% identical, preferably at least about 80% identical, and even more preferably at least about 95% identical to SEQ.ID.NO.:2 when measured by such standard programs as BLAST or FASTA. See, e.g.,Gish & States, 1993, Nature Genetics 3:266-272 and Altschul et al., 1990, J. Mol. Biol. 215:403-410 for examples of sequence comparison programs. For the purposes of this definition, examples of electrophysiological or pharmacological properties are: cation selectivity, voltage dependence of activation and inactivation, activation kinetics, reversal potential, and modulation by cyclic nucleotides such as cAMP or cGMP.

The present invention relates to the identification and cloning of DNA encoding the human HCN3 protein. Although cDNAs encoding mouse and rat HCN3 have been isolated, cDNA encoding the complete, correct human HCN3 protein has not previously been reported. A few ESTs, representing fragmentary sequences of human HCN3 (although not identified as HCN3 sequences) have been deposited in databanks. GenBank accession no. AI571225 represents an amino terminal fragment; AQ625620 represents a 3′ genomic sequence.

Other human HCN family members have been deposited. AF064876 represents a partial HCN1 sequence; AP065164 and AJ012582 represent HCN2; AJ132429 and AJ238850 represent HCN4.

Sequences from HCN family members of certain non-human species have been deposited in GenBank: AJ225123 (mouse HCN1); AJ247450 (rat HCN1); AP168122 (rabbit HCN1); AJ225122 (mouse HCN2); AJ225124 (mouse HCN3); AP247452 (rat HCN3); AF247453 (rat HCN4); AB022927 (rabbit HCN4).

The present invention provides DNA encoding human HCN3 having SEQ.ID.NO.:1. SEQ.ID.NO.:1 encodes a human HCN3 protein having SEQ.ID.NO.:2. Other sequence variants of human HCN3 have also been identified. Two single nucleotide polymorphisms (SNPs) were found in the cDNAs. They are highlighted and underlined below. In both cases, more than one clone was found containing each sequence. The resulting amino acids from these polymorphisms are also highlighted and underlined.

1. G (SEQ.ID.NO.:1) or C (SEQ.ID.NO.:3) at nucleotide position 1051 1051 (G/C)ACTCATCCA GTCCCTGGAC TCTTCCCGGC GTCAGTACCA GGAGAAGTAC (SEQ.ID.NO.:9)

resulting in G (SEQ.ID.NO.:2) or A (SEQ.ID.NO.:4) at amino acid position 348 301 ALFKAMSHML CIGYGQQAPV GMPDVWLTML SMIVGATCYA MFIGHAT(G/A)LI (SEQ.ID.NO.:10)

2. G (SEQ.ID.NO.:1) or T (SEQ.ID.NO.:5) at nucleotide position 1795 1751 GGCTCGGGGT GTTCGGGGTC GGGCCCCGAG CACAGGAGCT CAGC(G/T)TAGTG (SEQ.ID.NO.:11)

resulting in R (SEQ.ID.NO.:2) or L (SEQ.ID.NO.:6) at amino acid position 596 551 NSILQRKRSE PSPGSSGGIM EQHLVQHDRD MARGVRGRAP STGAQ(R/L)SGKP (SEQ.ID.NO.:12)

Northern blot analyses demonstrated expression of human HCN3 in a variety of tissues, including brain and heart. This pattern of expression suggests that the human HCN3 potassium channel subunit may have therapeutic relevance for the modulation of cellular excitability in the treatment of neurodegenerative diseases, cognitive and sensory disorders, inflammation, pain, cardiac brady- and tachy-arrhythmias, heart failure, ataxias, fertility disorders, hepatic dysfunction, pancreatic disorders (including diabetes), and diabetic neuropathy.

The present invention provides nucleic acids encoding the human HCN3 hyperpolarization-activated and cyclic nucleotide-gated cation channel that are substantially free from other nucleic acids. The nucleic acids may be DNA or RNA. The present invention also provides isolated and/or recombinant DNA molecules encoding the human HCN3 cation channel. The present invention provides DNA molecules substantially free from other nucleic acids as well as isolated and/or recombinant DNA molecules comprising the nucleotide sequence shown in SEQ.ID.NOs.:1, 3, or 5.

The present invention includes isolated DNA molecules as well as DNA molecules that are substantially free from other nucleic acids comprising the coding region of SEQ.ID.NOs.:1, 3, or 5. Accordingly, the present invention includes isolated DNA molecules and DNA molecules substantially free from other nucleic acids having a sequence comprising positions 9 to 2330 of SEQ.ID.NO.:1, 9 to 2330 of SEQ.ID.NO.:3, or 9 to 2330 of SEQ.ID.NO.:5.

Also included are recombinant DNA molecules having a nucleotide sequence comprising positions 9 to 2330 of SEQ.ID.NO.:1, 9 to 2330 of SEQ.ID.NO.:3, or 9 to 2330 of SEQ.ID.NO.:5. The novel DNA sequences of the present invention encoding the human HCN3 protein, in whole or in part, can be linked with other DNA sequences, i.e., DNA sequences to which DNA encoding the human HCN3 protein is not naturally linked, to form “recombinant DNA molecules” encoding the human HCN3 protein. Such other sequences can include DNA sequences that control transcription or translation such as, e.g., translation initiation sequences, internal ribosome entry sites, promoters for RNA polymerase II, transcription or translation termination sequences, enhancer sequences, sequences that control replication in microorganisms, sequences that confer antibiotic resistance, or sequences that encode a polypeptide “tag” such as, e.g., a polyhistidine tract, the FLAG epitope, or the myc epitope. The novel DNA sequences of the present invention can be inserted into vectors such as plasmids, cosmids, viral vectors, P1 artificial chromosomes, or yeast artificial chromosomes.

Included in the present invention are DNA sequences that hybridize to the reverse complement of SEQ.ID.NO:1 under conditions of high stringency. Preferably, these sequences encode proteins that have substantially the same biological activity as human HCN3 protein having SEQ.ID.NO.:2 and that have at least about 50%, preferably at least about 75%, and even more preferably at least about 95% nucleotide sequence identity with SEQ.ID.NO.:1. By way of example, and not limitation, a procedure using conditions of high stringency is as follows: Prehybridization of filters containing DNA is carried out for 2 hr. to overnight at 65° C. in buffer composed of 6×SSC, 5× Denhardt's solution, and 100 μg/ml denatured salmon sperm DNA. Filters are hybridized for 12 to 48 hrs at 65° C. in prehybridization mixture containing 100 μg/ml denatured salmon sperm DNA and 5-20×10⁶ cpm of ³²P-labeled probe. Washing of filters is done at 37° C. for 1 hr in a solution containing 2×SSC, 0.1% SDS. This is followed by a wash in 0.1×SSC, 0.1% SDS at 50° C. for 45 min. before autoradiography.

Other procedures using conditions of high stringency would include either a hybridization carried out in 5×SSC, 5×Denhardt's solution, 50% formamide at 42° C. for 12 to 48 hours or a washing step carried out in 0.2× SSPE, 0.2% SDS at 65° C. for 30 to 60 minutes.

Reagents mentioned in the foregoing procedures for carrying out high stringency hybridization are well know in the art. Details of the composition of these reagents can be found in, e.g., Sambrook, Fritsch, and Maniatis, 1989, Molecular Cloning: A Laboratory Manual, second edition, Cold Spring Harbor Laboratory Press. In addition to the foregoing, other conditions of high stringency which may be used are well known in the art.

The degeneracy of the genetic code is such that, for all but two amino acids, more than a single codon encodes a particular amino acid. This allows for the construction of synthetic DNA that encodes the human HCN3 protein where the nucleotide sequence of the synthetic DNA differs significantly from the nucleotide sequences of SEQ.ID.NO:1, SEQ.ID.NO:3, or SEQ.ID.NO:5 but still encodes the same human HCN3 protein as SEQ.ID.NO:1, SEQ.ID.NO:3, or SEQ.ID.NO:5. Such synthetic DNAs are intended to be within the scope of the present invention.

Mutated forms of SEQ.ID.NO:1, SEQ.ID.NO:3, or SEQ.ID.NO:5 are intended to be within the scope of the present invention. In particular, mutated forms of SEQ.ID.NO:1, SEQ.ID.NO:3, or SEQ.ID.NO:5 encoding a protein that forms cation channels having altered voltage sensitivity, current carrying properties, or other properties as compared to cation channels formed by the proteins encoded by SEQ.ID.NO:1, SEQ.ID.NO:3, or SEQ.ID.NO:5, are within the scope of the present invention. Such mutant forms can differ from SEQ.ID.NO:1, SEQ.ID.NO:3, or SEQ.ID.NO:5 by having nucleotide deletions, substitutions, or additions.

Also intended to be within the scope of the present invention are RNA molecules having sequences corresponding to SEQ.ID.NO:1, SEQ.ID.NO:3, or SEQ.ID.NO:5 or corresponding to the coding regions of SEQ.ID.NO:1, SEQ.ID.NO:3, or SEQ.ID.NO:5. The RNA molecules can be substantially free from other nucleic acids or can be isolated and/or recombinant RNA molecules.

Antisense nucleotides, DNA or RNA, that are the reverse complements of SEQ.ID.NO:1, SEQ.ID.NO:3, or SEQ.ID.NO:5, or portions thereof, are also within the scope of the present invention.

In addition, polynucleotides based on SEQ.ID.NO:1, SEQ.ID.NO:3, or SEQ.ID.NO:5 in which a small number of positions are substituted with non-natural or modified nucleotides such as inosine, methyl-cytosine, or deaza-guanosine are intended to be within the scope of the present invention. Polynucleotides of the present invention can also include sequences based on SEQ.ID.NO:1, SEQ.ID.NO:3, or SEQ.ID.NO:5 but in which non-natural linkages between the nucleotides are present. Such non-natural linkage can be, e.g. methylphosphonates, phosphorothioates, phosphorodithionates, phosphoroamidites, and phosphate esters. Polynucleotides of the present invention can also include sequences based on SEQ.ID.NO:1, SEQ.ID.NO:3, or SEQ.ID.NO:5 but having de-phospho linkages as bridges between nucleotides, e.g., siloxane, carbonate, carboxymethyl ester, acetamidate, carbamate, and thioether bridges. Other internucleotide linkages that can be present include N-vinyl, methacryloxyethyl, methacrylamide, or ethyleneimine linkages. Peptide nucleic acids based upon SEQ.ID.NO:1, SEQ.ID.NO:3, or SEQ.ID.NO:5 are also included in the present invention. Generally, such polynucleotides comprising non-natural or modified nucleotides and/or non-natural linkages between the nucleotides, as well as peptide nucleic acids, will encode the same, or highly similar, proteins as are encoded by SEQ.ID.NO:1, SEQ.ID.NO:3, or SEQ.ID.NO:5.

Another aspect of the present invention includes host cells that have been engineered to contain and/or express DNA sequences encoding the human HCN3 protein. Such recombinant host cells can be cultured under suitable conditions to produce human HCN3 protein. An expression vector comprising DNA encoding human HCN3 protein can be used for the expression of human HCN3 protein in a recombinant host cell. Recombinant host cells may be prokaryotic or eukaryotic, including but not limited to, bacteria such as E. coli, fungal cells such as yeast, mammalian cells including, but not limited to, cell lines of human, bovine, porcine, monkey and rodent origin, amphibian cells such as Xenopus oocytes, and insect cells including but not limited to Drosophila and silkworm derived cell lines (e.g., Spodoptera frugiperda). Cells and cell lines which are suitable for recombinant expression of human HCN3 protein and which are widely available, include but are not limited to, L cells L-M(TK⁻) (ATCC CCL 1.3), L cells L-M (ATCC CCL 1.2), HEK 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C1271 (ATCC CRL 1616), BS-C-1 (ATCC CCL 26), MRC-5 (ATCC CCL 171), CPAE (ATCC CCL 209), Saos-2 (ATCC HTB-85), ARPE-19 human retinal pigment epithelium (ATCC CRL-2302), Xenopus melanophores, and Xenopus oocytes.

A variety of mammalian expression vectors can be used to express recombinant human HCN3 protein in mammalian cells. Commercially available mammalian expression vectors which arc suitable include, but are not limited to, pMC1neo (Stratagene), pSG5 (Stratagene), pcDNAI and pcDNAIamp, pcDNA3, pcDNA3.1, pCR3.1 (Invitrogen), EBO-pSV2-neo (ATCC 37593), pBPV-1(8-2) (ATCC 37110), pdBPV-MMTneo(342-12) (ATCC 37224), pRSVgpt (ATCC 37199), pRSVneo (ATCC 37198), pIZD35 (ATCC 37565), and pSV2-dhfr (ATCC 37146). Another suitable vector is the PT7TS oocyte expression vector.

Following expression in recombinant cells, human HCN3 protein can be purified by conventional techniques to a level that is substantially free from other proteins. Techniques that can be used include ammonium sulfate precipitation, hydrophobic or hydrophilic interaction chromatography, ion exchange chromatography, affinity chromatography, phosphocellulose chromatography, size exclusion chromatography, preparative gel electrophoresis, and alcohol precipitation. In some cases, it may be advantageous to employ protein denaturing and/or refolding steps in addition to such techniques.

Certain ion channel subunit proteins have been found to require the expression of other ion channel subunits in order to be properly expressed at high levels and inserted in membranes. For example, co-expression of KCNQ3 appears to enhance the expression of KCNQ2 in Xenopus oocytes (Wang et al., 1998, Science 282:1890-1893). Also, some voltage-gated potassium channel Kvα subunits require other related a subunits or Kvβ subunits (Shi et al., 1995, Neuron 16:843-852). Accordingly, the recombinant expression of human HCN3 proteins may under certain circumstances benefit from the co-expression of other ion channel proteins and such co-expression is intended to be within the scope of the present invention. Such co-expression can be effected by transfecting an expression vector encoding human HCN3 protein into a cell that naturally expresses another ion channel protein. Alternatively, an expression vector encoding human HCN3 protein can be transfected into a cell in which an expression vector encoding another ion channel protein has also been transfected. Preferably, such a cell does not naturally express human HCN3 subunit proteins or the other ion channel protein. Co-expression of human HCN3 with other HCN family proteins such as HCN1, HCN2, or HCN4 may be of benefit. In addition, since these cation channels are also modulated by cyclic nucleotides, co-expression of HCN3 with other types of receptors, such as those that control levels of intracellular cyclic nucleotides (e.g., the beta adrenergic receptor) may also be of benefit and is also within the scope of the present invention.

The present invention includes human HCN3 proteins substantially free from other proteins. The amino acid sequences of full-length human HCN3 subunit proteins are shown in SEQ.ID.NOs.:2, 4, and 6. Thus, the present invention includes human HCN3 protein substantially free from other proteins comprising an amino acid sequence selected from the group consisting of SEQ.ID.NOs.:2, 4, and 6. The present invention also includes isolated human HCN3 protein comprising an amino acid sequence selected from the group consisting of SEQ.ID.NOs.:2, 4, and 6.

Mutated forms of human HCN3 proteins are intended to be within the scope of the present invention. In particular, mutated forms of SEQ.ID.NOs:2, 4, or 6 that form cation channels having altered electrophysiological or pharmacological properties as compared to potassium channels formed by SEQ.ID.NOs:2, 4, or 6 are within the scope of the present invention.

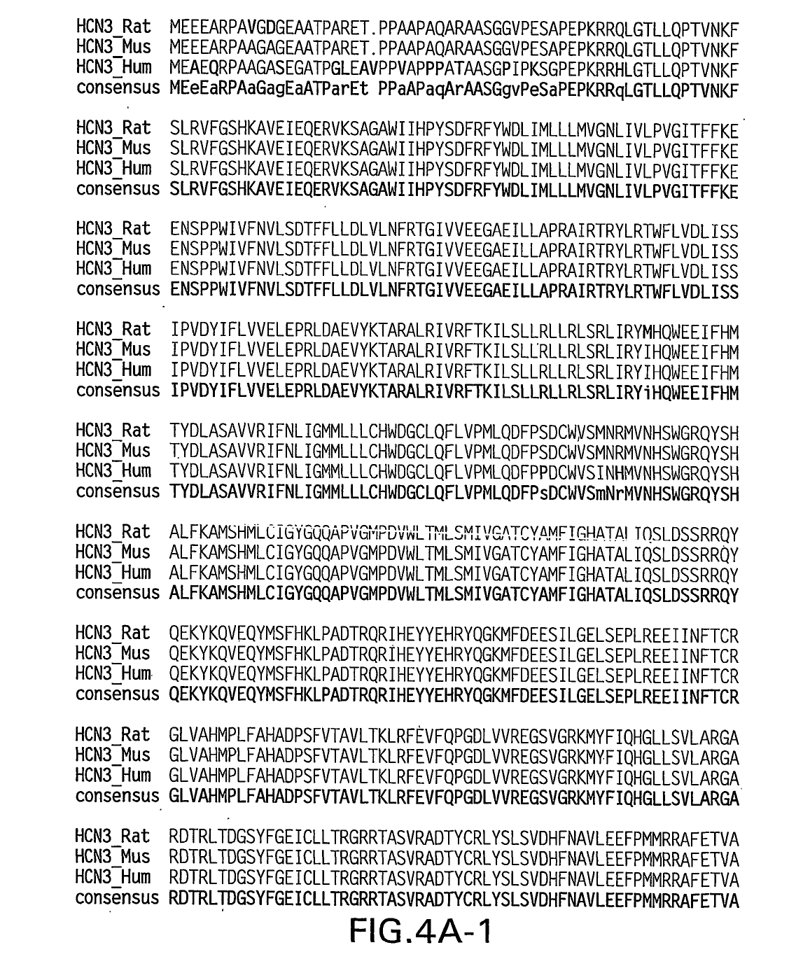

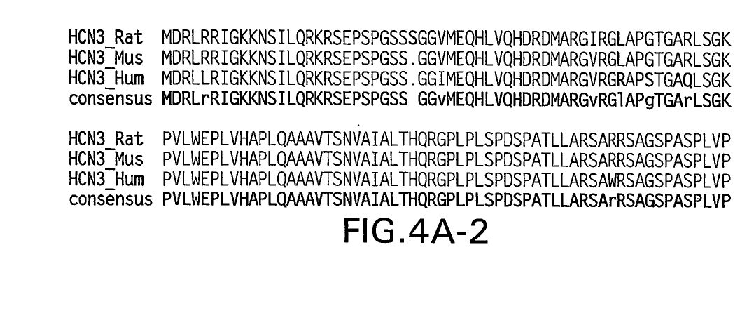

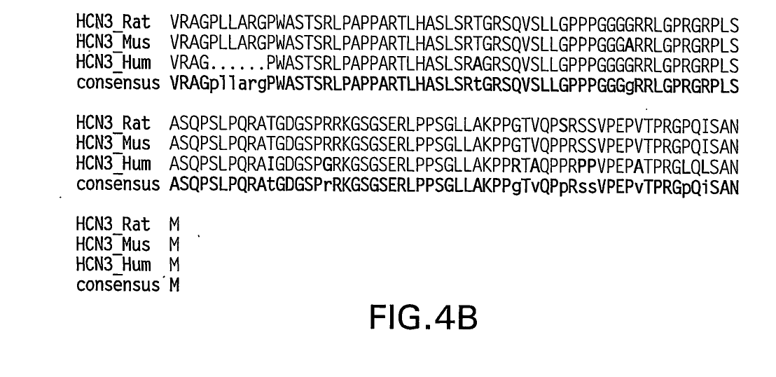

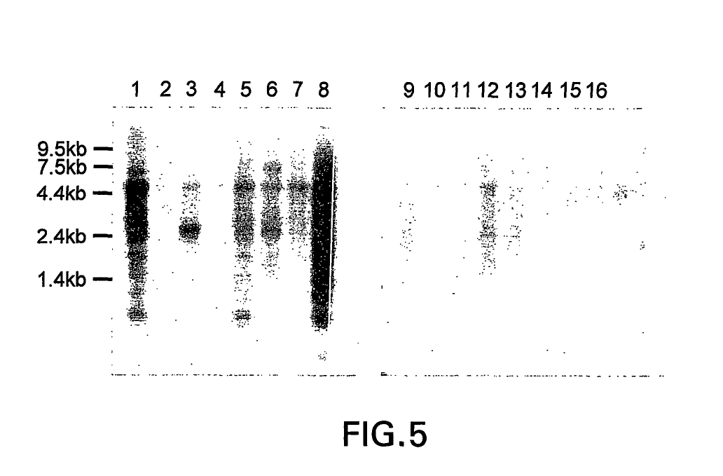

As with many proteins, it may be possible to modify many of the amino acids of the human HCN3 protein and still retain substantially the same biological activity as for the original protein. Thus, the present invention includes modified human HCN3 proteins which have amino acid deletions, additions, or substitutions but that still retain substantially the same biological activity as naturally occurring human HCN3 proteins. It is generally accepted that single amino acid substitutions do not usually alter the biological activity of a protein (see, e.g., Molecular Biology of the Gene, Watson et al, 1987, Fourth Ed., The Benjamin/Cummings Publishing Co., Inc., page 226; and Cunningham & Wells, 1989, Science 244:1081-1085). Accordingly, the present invention includes polypeptides where one amino acid substitution has been made in SEQ.ID.NOs:2, 4, or 6 wherein the polypeptides still retain substantially the same biological activity as naturally occurring human HCN3 proteins. The present invention also includes polypeptides where two or more amino acid substitutions have been made in SEQ.ID.NOs:2, 4, or 6 wherein the polypeptides still retain substantially the same biological activity as naturally occurring human HCN3 proteins. In particular, the present invention includes embodiments where the above-described substitutions are conservative substitutions. In particular, the present invention includes embodiments where the above-described substitutions do not occur in conserved positions. Conserved positions are those positions in which the human HCN3 protein having SEQ.ID.NO:2, the mouse HCN3 protein (SEQ.ID.NO:7), and the rat HCN3 protein (SEQ.ID.NO:8) share the same amino acid (see FIG. 4).

The human HCN3 proteins of the present invention may contain post-translational modifications, e.g., covalently linked carbohydrate, phosphorylation, myristoylation, palmytoylation.

The present invention also includes chimeric human HCN3 proteins. Chimeric human HCN3 proteins consist of a contiguous polypeptide sequence of at least a portion of a human HCN3 protein fused to a polypeptide sequence that is not from a human HCN3 protein. The portion of the human HCN3 protein must include at least 10, preferably at least 25, and most preferably at least 50 contiguous amino acids from SEQ.ID.NO:2, 4, or 6.

The present invention also includes isolated human HCN3 protein and isolated DNA encoding human HCN3 protein. Use of the term “isolated” indicates that the human HCN3 protein or DNA has been removed from its normal cellular environment. Thus, an isolated human HCN3 protein may be in a cell-free solution or placed in a different cellular environment from that in which it occurs naturally. The term isolated does not necessarily imply that an isolated human HCN3 protein is the only, or predominant, protein present (although that is one of the meanings of isolated), but instead means that the isolated human HCN3 protein is at least 95% free of non-amino acid material (e.g., nucleic acids, lipids, carbohydrates) naturally associated with the human HCN3 protein.

It is known that certain ion channel subunits can interact to form heteromeric complexes resulting in functional ion channels. For example, KCNQ2 and KCNQ3 can assemble to form a heteromeric functional potassium channel (Wang et al., 1998, Science 282:1890-1893). Accordingly, it is believed that the human HCN3 proteins of the present invention may also be able to form heteromeric structures with other proteins where such heteromeric structures form functional ion channels. Thus, the present invention includes such heteromers comprising human HCN3 protein. Preferred heteromers are those in which the human HCN3 protein forms heteromers with at least one other HCN family member, e.g., HCN1, HCN2, or HCN4. Preferably, the other HCN family member is a human HCN family member.

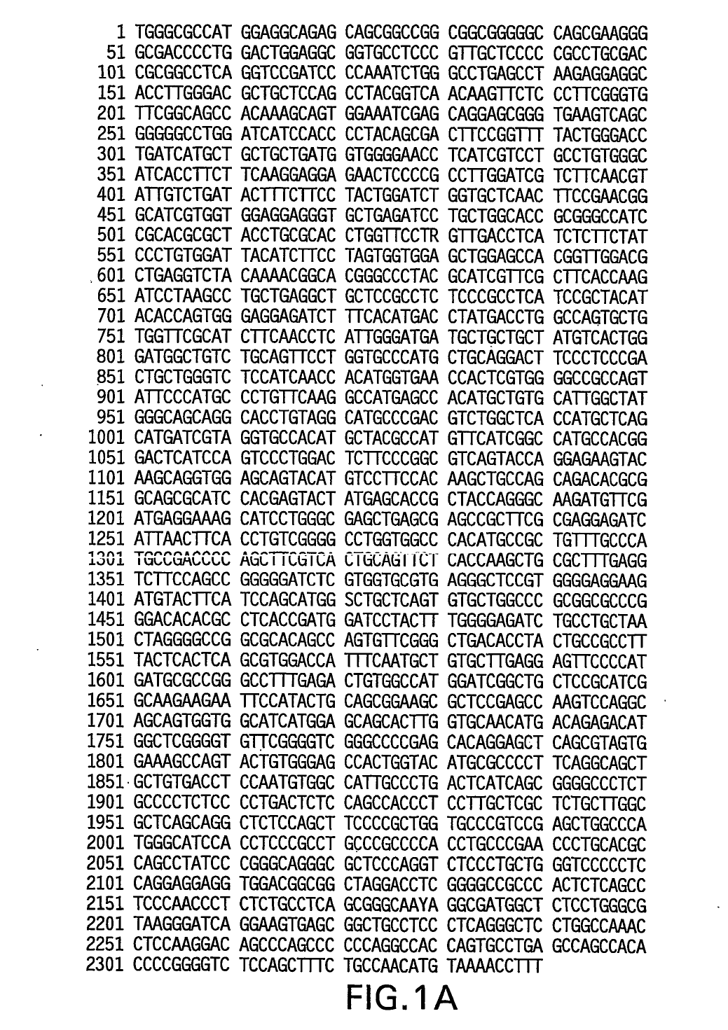

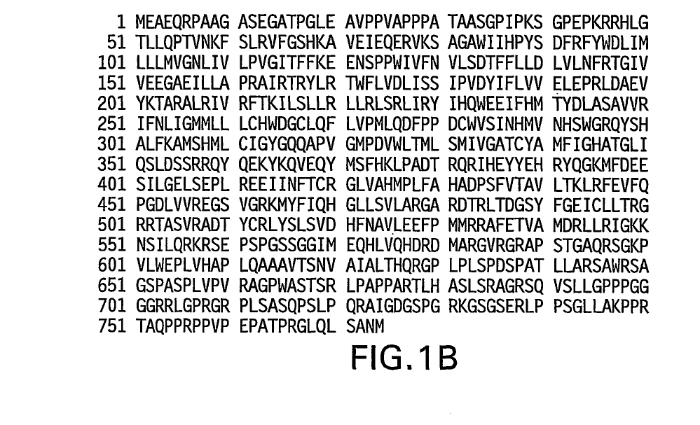

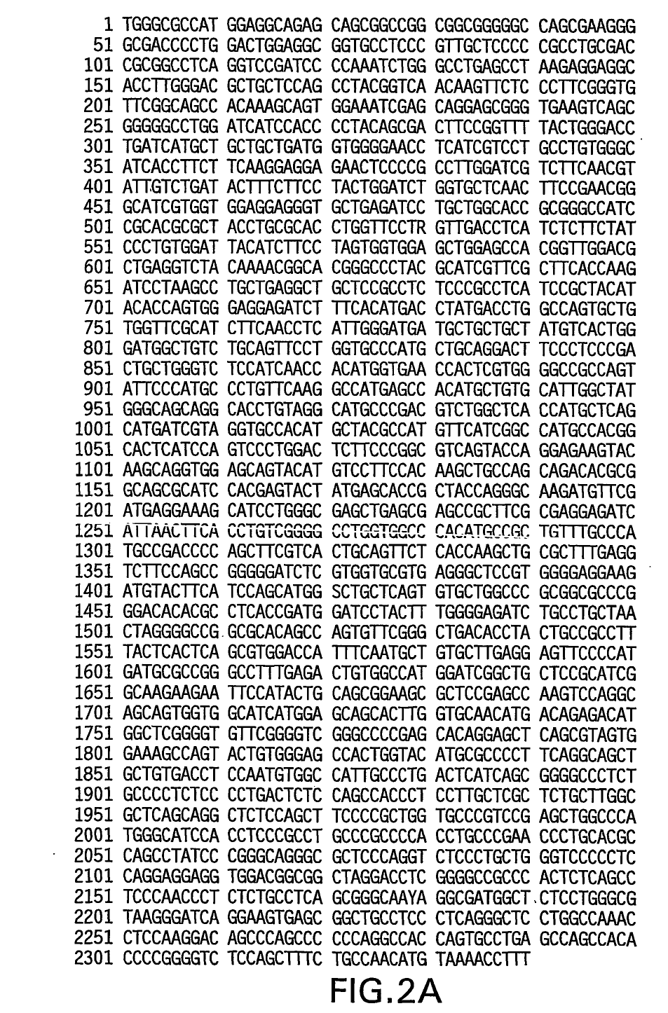

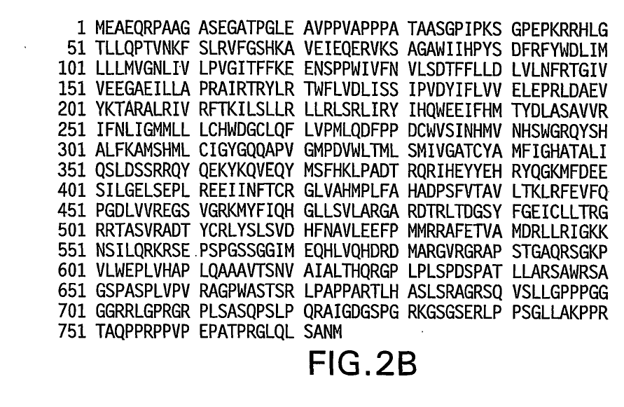

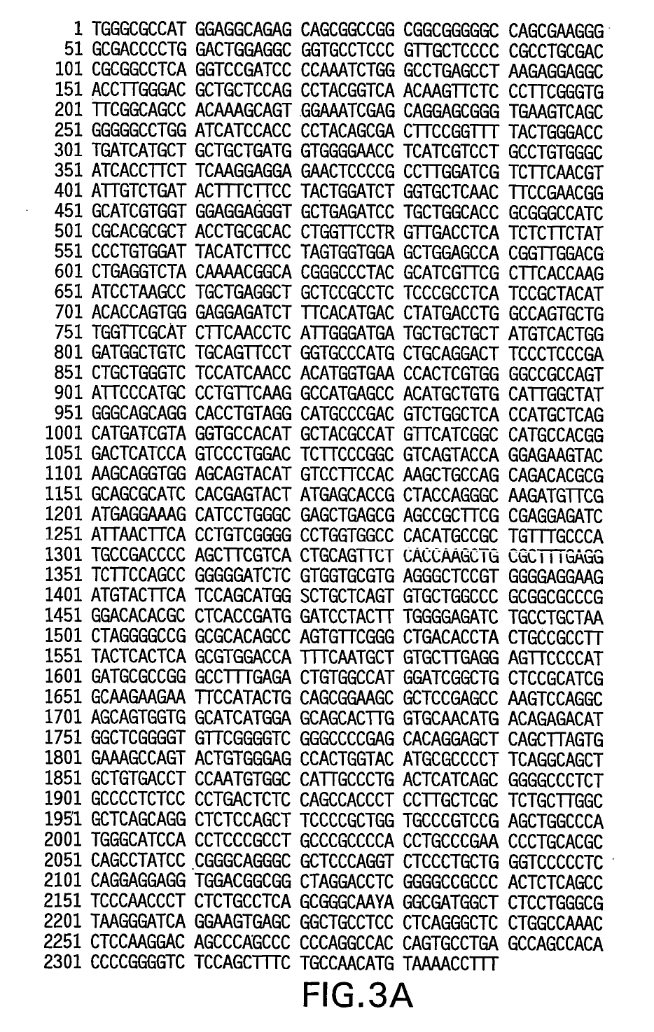

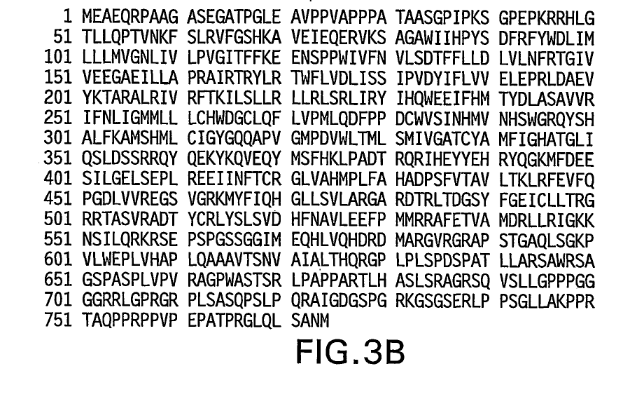

DNA encoding human HCN3 proteins can be obtained by methods well known in the art. For example, a cDNA fragment encoding full-length human HCN3 protein can be isolated from human brain or heart cDNA by using the polymerase chain reaction (PCR) employing suitable primer pairs. Such primer pairs can be selected based upon the DNA sequences encoding the human HCN3 proteins shown in FIGS. 1-3 as SEQ.ID.NOs.:1, 3, and 5. Suitable primer pairs would be, e.g.:

5′ TGGGCGCCAT GGAGGCAGAGC 3′ (SEQ.ID.NO.:13)

5′ AAAGGTTTTACATGTTGGC 3′ (SEQ.ID.NO.:14)

The above primers are meant to be illustrative only; one skilled in the art would readily be able to design other suitable primers based upon SEQ.ID.NOs.:1, 3, and 5. Such primers could be produced by methods of oligonucleotide synthesis that are well known in the art.

PCR reactions can be carried out with a variety of thermostable enzymes including but not limited to AmpliTaq, AmpliTaq Gold, or Vent polymerase. For AmpliTaq, reactions can be carried out in 10 mM Tris-Cl, pH 8.3, 2.0 mM MgCl₂, 200 μM of each dNTP, 50 mM KCl, 0.2 μM of each primer, 10 ng of DNA template, 0.05 units/μl of AmpliTaq. The reactions are heated at 95° C. for 3 minutes and then cycled 35 times using the cycling parameters of 95° C., 20 seconds, 62° C., 20 seconds, 72° C., 3 minutes. In addition to these conditions, a variety of suitable PCR protocols can be found in PCR Primer, A Laboratory Manual, edited by C. W. Dieffenbach and G. S. Dveksler, 1995, Cold Spring Harbor Laboratory Press; or PCR Protocols: A Guide to Methods and Applications, Michael et al., eds., 1990, Academic Press.

Since the human HCN3 proteins of the present invention are homologous to other cation channel subunit proteins, it is desirable to sequence the clones obtained by the herein-described methods, in order to verify that the desired human HCN3 protein has in fact been obtained. Sequencing is also advisable in order to ensure that one has obtained the desired cDNA from among SEQ.ID.NO:1, 3, and 5.

By these methods, cDNA clones encoding human HCN3 proteins can be obtained. These cDNA clones can be cloned into suitable cloning vectors or expression vectors, e.g., the mammalian expression vector pcDNA3.1 (Invitrogen, San Diego, Calif.). Human HCN3 protein can then be produced by transferring expression vectors encoding human HCN3 or portions thereof into suitable host cells and growing the host cells under appropriate conditions. Human HCN3 protein can then be isolated by methods well know in the art.

As an alternative to the above-described PCR methods, cDNA clones encoding human HCN3 proteins can be isolated from cDNA libraries using as a probe oligonucleotides specific for human HCN3 and methods well known in the art for screening cDNA libraries with oligonucleotide probes. Such methods are described in, e.g., Sambrook et al., 1989, Molecular Cloning: A Laboratory Manual; Cold Spring Harbor Laboratory, Cold Spring Harbor, New York; Glover, D. M. (ed.), 1985, DNA Cloning: A Practical Approach, MRL Press, Ltd., Oxford, U.K., Vol. I, II. Oligonucleotides that are specific for human HCN3 and that can be used to screen cDNA libraries can be readily designed based upon the DNA sequences shown in FIGS. 1-3 (viz., SEQ.ID.NOs:1, 3, and 5) and can be synthesized by methods well-known in the art.

Genomic clones containing the human HCN3 gene can be obtained from commercially available human PAC or BAC libraries from suppliers such as, e.g., Research Genetics, Huntsville, Ala. Alternatively, one may prepare genomic libraries, e.g., in P1 artificial chromosome vectors, from which genomic clones containing the human HCN3 gene can be isolated, using probes based upon the human HCN3 DNA sequences disclosed herein. Methods of preparing such libraries are known in the art (see, e.g., Ioannou et al., 1994, Nature Genet. 6:84-89).

The novel DNA sequences of the present invention can be used in various diagnostic methods. The present invention provides diagnostic methods for determining whether a patient carries a mutation in the human HCN3 gene. In broad terms, such methods comprise determining the DNA sequence of a region in or near the human HCN3 gene from the patient and comparing that sequence to the sequence from the corresponding region of the human HCN3 gene from a non-affected person, i.e., a person who does not have the condition which is being diagnosed, where a difference in sequence between the DNA sequence of the gene from the patient and the DNA sequence of the gene from the non-affected person indicates that the patient has a mutation in the human HCN3 gene.

The present invention also provides oligonucleotide probes, based upon SEQ.ID.NOs:1, 3, or 5 that can be used in diagnostic methods to identify patients having mutated forms of the human HCN3 gene, to determine the level of expression of RNA encoding human HCN3, or to isolate genes homologous to human HCN3 from other species. In particular, the present invention includes DNA oligonucleotides comprising at least about 10, 15, or 18 (but not more than 100) contiguous nucleotides of SEQ.ID.NOs:1, 3, or 5 where the oligonucleotide probe comprises no stretch of contiguous nucleotides longer than 5 from: SEQ.ID.NOs:1, 3, or 5 other than the said at least about 10, 15, or 18 contiguous nucleotides. The oligonucleotides can be substantially free from other nucleic acids. Also provided by the present invention are corresponding RNA oligonucleotides. The DNA or RNA oligonucleotides can be packaged in kits.

The present invention makes possible the recombinant expression of human HCN3 protein in various cell types. Such recombinant expression facilitates the study of this protein so that its biochemical activity and its possible role in various diseases such as neurodegenerative diseases, cognitive and sensory disorders, pain, cardiac brady- and tachy-arrhythmias, ataxias, fertility disorders, hepatic dysfunction, pancreatic disorders (including diabetes), inflammation, heart failure, and diabetic neuropathy can be elucidated.

The present invention also makes possible the development of assays which measure the biological activity of cation channels containing human HCN3 protein. Assays using recombinantly expressed human HCN3 protein are especially of interest. Such assays can be used to screen libraries of compounds or other sources of compounds to identify compounds that are activators or inhibitors of the activity of cation channels containing human HCN3 protein. Such identified compounds can serve as “leads” for the development of pharmaceuticals that can be used to treat patients having diseases in which it is beneficial to enhance or suppress cation channel activity.

In versions of the above-described assays, cation channels containing mutant human HCN3 proteins are used and inhibitors or activators of the activity of the mutant cation channels are identified.

Preferred cell lines for recombinant expression of human HCN3 proteins are those which do not express endogenous cation channels. Cell lines expressing recombinant human HCN3 can be exposed to and loaded with ⁸⁶Rb, an ion which can substitute for potassium in many ion channels. The efflux of ⁸⁶Rb out of such cells can be assayed in the presence and absence of collections of substances (e.g., combinatorial libraries, natural products, analogues of lead compounds produced by medicinal chemistry), or members of such collections, and those substances that are able to alter ⁸⁶Rb efflux thereby identified. Such substances are likely to be activators or inhibitors of cation channels containing human HCN3 protein.

Activators and inhibitors of cation channels containing human HCN3 proteins are likely to be substances that are capable of binding to cation channels containing human HCN3 proteins. Thus, one type of assay determines whether one or more of a collection of substances is capable of such binding.

Accordingly, the present invention provides a method of identifying substances that bind to cation channels containing human HCN3 protein comprising:

(a) providing cells expressing a cation channel containing human HCN3 protein;

(b) exposing the cells to a substance that is not known to bind cation channels containing human HCN3 protein;

(c) determining the amount of binding of the substance to the cells;

(d) comparing the amount of binding in step (c) to the amount of binding of the substance to control cells where the control cells are substantially identical to the cells of step (a) except that the control cells do not express human HCN3 protein;

where if the amount of binding in step (c) is greater than the amount of binding of the substance to control cells, then the substance binds to cation channels containing human HCN3 protein.

An example of control cells that are substantially identical to the cells of step (a) would be a parent cell line where the parent cell line is transfected with an expression vector encoding human HCN3 protein in order to produce the cells expressing a cation channel containing human HCN3 protein of step (a).

Another version of this assay makes use of compounds that are known to bind to cation channels containing human HCN3 protein. Substances that are new binders are identified by virtue of their ability to augment or block the binding of these known compounds. This can be done if the known compound is used at a concentration that is far below saturation, in which case a substance that is a new binder is likely to be able to either augment or block the binding of the known compound. Substances that have this ability are likely themselves to be inhibitors or activators of cation channels containing human HCN3 protein.

According, the present invention includes a method of identifying substances that bind cation channels containing human HCN3 protein and thus are likely to be inhibitors or activators of cation channels containing human HCN3 protein comprising:

(a) providing cells expressing cation channels containing human HCN3 protein;

(b) exposing the cells to a compound that is known to bind to the cation channels containing human HCN3 protein in the presence and in the absence of a substance not known to bind to cation channels containing human HCN3 protein;

(c) determining the amount of binding of the compound to the cells in the presence and in the absence of the substance;

where if the amount of binding of the compound in the presence of the substance differs from that in the absence of the substance, then the substance binds cation channels containing human HCN3 protein and is likely to be an inhibitor or activator of cation channels containing human HCN3 protein.

Generally, the known compound is labeled (e.g., radioactively, enzymatically, fluorescently) in order to facilitate measuring its binding to the cation channels.

Once a substance has been identified by the above-described methods, it can be assayed in functional tests, such as those described herein, in order to determine whether it is an inhibitor or an activator.

In particular embodiments, the compound known to bind potassium channels containing human HCN3 protein is selected from the group consisting of: ZD7288 and L-cis-diltiazem.

The present invention includes a method of identifying activators or inhibitors of cation channels containing human HCN3 protein comprising:

(a) recombinantly expressing human HCN3 protein in a host cell so that the recombinantly expressed human HCN3 protein forms cation channels either by itself or by forming heteromers with other cation channel subunit proteins;

(b) measuring the biological activity of the cation channels formed in step (a) in the presence and in the absence of a substance not known to be an activator or an inhibitor of cation channels containing human HCN3 protein;

where a change in the biological activity of the cation channels formed in step (a) in the presence as compared to the absence of the substance indicates that the substance is an activator or an inhibitor of cation channels containing human HCN3 protein.

In particular embodiments of the methods described herein, the biological activity is the conduction of a mixed Na⁺/K⁺ current or the efflux of ⁸⁶Rb.

In particular embodiments, it may be advantageous to recombinantly express the other subunits of cation channels. Alternatively, it may be advantageous to use host cells that endogenously express such other subunits. Other subunits may be other HCN family members such as HCN1, HCN2, or HCN4, particularly other human HCN family members.

In particular embodiments, a vector encoding human HCN3 protein is transferred into Xenopus oocytes in order to cause the expression of human HCN3 protein in the oocytes. Alternatively, RNA encoding human HCN3 protein can be prepared in vitro and injected into the oocytes, also resulting in the expression of human HCN3 protein in the oocytes. Following expression of the human HCN3 protein in the oocytes, and following the formation of cation channels containing human HCN3, membrane currents are measured after the transmembrane voltage is changed in steps. A change in membrane current is observed when the cation channels containing human HCN3 open or close, modulating sodium and potassium ion flow. Similar studies were reported for KCNQ2 and KCNQ3 potassium channels in Wang et al., 1998, Science 282:1890-1893 and for MinK channels by Goldstein & Miller, 1991, Neuron 7:403-408. These references and references cited therein can be consulted for guidance as to how to carry out such studies. In such studies it may be advantageous to co-express other cation channel subunit proteins (e.g., HCN1, HCN2, or HCN4) in addition to human HCN3 in the oocytes.

Inhibitors or activators of cation channels containing human HCN3 protein can be identified by exposing the oocytes to individual substances or collections of substances and determining whether the substances can block/diminish or enhance the membrane currents observed in the absence of the substance.

Accordingly, the present invention provides a method of identifying inhibitors or activators of cation channels containing human HCN3 protein comprising:

(a) expressing human HCN3 protein in cells such that cation channels containing human HCN3 protein are formed;

(b) changing the transmembrane potential of the cells in step (a) from a potential where the cation channels containing human HCN3 protein are closed to a potential where cation channels containing human HCN3 protein are open in the presence and the absence of a substance not known to be an inhibitor or an activator of cation channels containing human HCN3 protein;

(c) measuring mixed sodium/potassium currents following step (b);

where if the mixed sodium/potassium currents measured in step (c) are less in the presence rather than in the absence of the substance, then the substance is an inhibitor of cation channels containing human HCN3 protein;

where if the mixed sodium/potassium currents measured in step (c) are greater in the presence rather than in the absence of the substance, then the substance is an activator of cation channels containing human HCN3 protein.

In general, for step (b), the potential where the cation channels containing human HCN3 protein are closed will be a depolarized potential and the potential where cation channels containing human HCN3 protein are open will be a hyperpolarized potential.

The method described above can be practiced by the use of techniques that are well known in the art such as voltage clamp studies or patch clamp studies. Alternatively, where the cells contain a β-adrenergic receptor as well as the HCN3 channel, instead of changing the membrane potential by voltage clamp to turn on the HCN3 current, the potential can be held steady and a β-adrenergic receptor agonist can be added to the cells. This should increase cAMP concentration and turn on the HCN3 channel. One could then assay for activators and inhibitors in the same way as above by looking at the currents plus/minus the compounds.

The present invention also includes assays for the identification of activators and inhibitors of cation channels containing human HCN3 protein that are based upon fluorescence resonance energy transfer (FRET) between a first and a second fluorescent dye where the first dye is bound to one side of the plasma membrane of a cell expressing cation channels containing human HCN3 protein and the second dye is free to shuttle from one face of the membrane to the other face in response to changes in membrane potential. In certain embodiments, the first dye is impenetrable to the plasma membrane of the cells and is bound predominately to the extracellular surface of the plasma membrane. The second dye is trapped within the plasma membrane but is free to diffuse within the membrane. At polarized (i.e., negative) resting potentials of the membrane, the second dye is bound predominately to the inner surface of the extracellular face of the plasma membrane, thus placing the second dye in close proximity to the first dye. This close proximity allows for the generation of a large amount of FRET between the two dyes. At depolarized potentials, the second dye moves from the extracellular face of the membrane to the intracellular face, thus increasing the distance between the dyes. This increased distance results in a decrease in FRET, with a corresponding increase in fluorescent emission derived from the first dye and a corresponding decrease in the fluorescent emission from the second dye. In this way, the amount of FRET between the two dyes can be used to measure the polarization state of the membrane. For a description of this technique, see González & Tsien, 1997, Chemistry & Biology 4:269-277. See also González & Tsien, 1995, Biophys. J. 69:1272-1280 and U.S. Pat. No. 5,661,035.

In certain embodiments, the first dye is a fluorescent lectin or a fluorescent phospholipid that acts as the fluorescent donor. Examples of such a first dye are: a coumarin-labeled phosphatidylethanolamine (e.g., N-(6-chloro-7-hydroxy-2-oxo-2H-1-benzopyran-3-carboxamidoacetyl)-dimyristoylphosphatidyl-ethanolamine) or N-(7-nitrobenz-2-oxa-1,3-diazol-4-yl)-dipalmitoylphosphatidylethanolamine); a fluorescently-labeled lectin (e.g., fluorescein-labeled wheat germ agglutinin). In certain embodiments, the second dye is an oxonol that acts as the fluorescent acceptor. Examples of such a second dye are: bis(1,3-dialkyl-2-thiobarbiturate)trimethineoxonols (e.g., bis(1,3-dihexyl-2-thiobarbiturate)trimethineoxonol) or pentamethineoxonol analogues (e.g., bis(1,3-dihexyl-2-thiobarbiturate)pentamethineoxonol; or bis(1,3-dibutyl-2-thiobarbiturate)pentamethineoxonol). See González & Tsien, 1997, Chemistry & Biology 4:269-277 for methods of synthesizing various dyes suitable for use in the present invention. In certain embodiments, the assay may comprise a natural carotenoid, e.g., astaxanthin, in order to reduce photodynamic damage due to singlet oxygen.

The above described assays can be utilized to discover activators and inhibitors of cation channels containing human HCN3 protein. Such assays will generally utilize cells that express cation channels containing human HCN3 protein, e.g., by transfection with expression vectors encoding human HCN3 protein and, optionally, other cation channel subunits.

The cellular membrane potential is determined by the balance between inward (depolarizing) and outward (repolarizing) ionic fluxes through various ion pumps and channels. FRET based assays could be developed by co-expressing HCN3 containing cation channels with an inward rectifier potassium channel. The inward rectifier will allow potassium efflux from the cell, which tends to stabilize the membrane potential near the potassium equilibrium potential, E_{K}, (typically about −80 mV). When human HCN3 is expressed in cells having a resting membrane potential lower than about −30 mV, especially cells having resting membrane potentials lower than about −50 to −70 mV, the channels formed by human HCN3 will be open and will tend to pass a cation current into the cell, thus tending to depolarize the membrane potential. The presence of an inhibitor of a cation channel containing human HCN3 will prevent, or diminish, the ability of HCN3 to depolarize the membrane potential. Thus, membrane potential will remain negative (i.e., hyperpolarized) in the presence of human HCN3 inhibitors. Such changes in membrane potential that are caused by inhibitors of potassium channels containing human HCN3 protein can be monitored by the assays using FRET described above.

Accordingly, the present invention provides a method of identifying inhibitors of cation channels containing human HCN3 protein comprising:

(a) providing cells comprising:

(1) an expression vector that directs the expression of human HCN3 protein in the cells so that cation channels containing human HCN3 protein are formed in the cells and where the cells have a resting membrane potential lower than about −30 mV;

(2) a first fluorescent dye, where the first dye is bound to one side of the plasma membrane of the cells; and

(3) a second fluorescent dye, where the second fluorescent dye is free to distribute from one face of the plasma membrane of the cells to the other face in response to changes in membrane potential;

(b) exposing the cells to a substance;

(c) measuring the amount of fluorescence resonance energy transfer (FRET) in the cells in the presence and in the absence of the substance;

(d) comparing the amount of FRET exhibited by the cells in the presence and in the absence of the substance;

where if the amount of FRET exhibited by the cells in the presence of the substance is greater than the amount of FRET exhibited by the cells in the absence of the substance then the substance is an inhibitor of potassium channels containing human HCN3 protein.

If the cells are exposed to a substance that is an activator (rather than an inhibitor) of cation channels containing human HCN3 protein, then the HCN3 channels will pass more current into the cell, tending to move the membrane potential to a more positive (i.e., depolarized) level. This depolarization can also be monitored by the FRET assays described above.

Accordingly, the present invention provides a method of identifying activators of cation channels containing human HCN3 protein comprising:

(a) providing cells comprising:

(1) an expression vector that directs the expression of human HCN3 protein in the cells so that cation channels containing human HCN3 protein are formed in the cells and where the cells have a resting membrane potential lower than about −30 mV;

(2) a first fluorescent dye, where the first dye is bound to one side of the plasma membrane of the cells; and

(3) a second fluorescent dye, where the second fluorescent dye is free to distribute from one face of the plasma membrane of the cells to the other face in response to changes in membrane potential;

(b) exposing the cells to a substance;

(c) measuring the amount of fluorescence resonance energy transfer (FRET) in the cells in the presence and in the absence of the substance;

(d) comparing the amount of FRET exhibited by the cells in the presence and in the absence of the substance;

where if the amount of FRET exhibited by the cells in the presence of the substance is less than the amount of FRET exhibited by the cells in the absence of the substance then the substance is an inhibitor of potassium channels containing human HCN3 protein.

As an alternative way of ensuring that the ion channels containing human HCN3 protein are turned on, one can utilize cells containing a β-adrenergic receptor and expose those cells to an agonist of the β-adrenergic receptor. This will cause an increase in cAMP concentration in the cells and thus opening the ion channels containing human HCN3 protein. Further exposing such cells to substances that are inhibitors of ion channels containing human HCN3 protein will close those channels, leading to a hyperpolarization of the cells' membrane potentials. This hyperpolarization can be measured by FRET-based assays.

Accordingly, the present invention includes a method of identifying inhibitors of ion channels containing human HCN3 protein comprising:

(a) providing cells comprising:

(1) an expression vector that directs the expression of human HCN3 protein in the cells so that ion channels containing human HCN3 protein are formed in the cells;

(2) a β-adrenergic receptor;

(3) a first fluorescent dye, where the first dye is bound to one side of the plasma membrane of the cells; and

(4) a second fluorescent dye, where the second fluorescent dye is free to distribute from one face of the plasma membrane of the cells to the other face in response to changes in membrane potential;

(b) exposing the cells to an agonist of the β-adrenergic receptor so that the cAMP concentration in the cells increases to a level such that the cation channels containing human HCN3 protein are open;

(c) exposing the cells to a substance;

(d) measuring the amount of fluorescence resonance energy transfer (FRET) in the cells in the presence and in the absence of the substance;

(e) comparing the amount of FRET exhibited by the cells in the presence and in the absence of the substance;

where if the amount of FRET exhibited by the cells in the presence of the substance is greater than the amount of FRET exhibited by the cells in the absence of the substance then the substance is an inhibitor of ion channels containing human HCN3 protein.

In particular embodiments of the above-described methods, the cells also express an inward rectifier potassium channel, either endogenously (e.g., RBL cells) or recombinantly (e.g., as a result of having been transfected with an expression vector encoding the inward rectifier potassium channel). In such embodiments, it is desirable to perform control experiments to rule out the possibility that the substances identified are actually agonists of the inward rectifier potassium channel rather than inhibitors of potassium channels containing human HCN3 protein. This can be done by expressing the HCN3 protein or the inward rectifier potassium channel individually in cells and testing the effect of the substances on the HCN3 protein and the inward rectifier potassium channel by patch clamp techniques.

As another type of control experiment, in order to be sure that the effect of the substance in the above-described assays is arising through its action at cation channels containing human HCN3 protein, experiments can be run in which the cells are as above, except that they do not contain an expression vector that directs the expression of human HCN3 protein.

In particular embodiments of the above-described methods, the expression vectors are transfected into the test cells.

In particular embodiments of the above-described methods, the human HCN3 protein has an amino acid sequence selected from the group consisting of SEQ.ID.NOs.:2, 4, and 6. In particular embodiments of the above-described methods, the expression vector comprises positions 9 to 2330 of SEQ.ID.NO.:1, 9 to 2330 of SEQ.ID.NO.:3, or 9 to 2330 of SEQ.ID.NO.:5.

In particular embodiments of the above-described methods, the first fluorescent dye is selected from the group consisting of: a fluorescent lectin; a fluorescent phospholipid; a coumarin-labeled phosphatidylethanolamine; N-(6-chloro-7-hydroxy-2-oxo-2H-1-benzopyran-3-carboxamidoacetyl)-dimyristoylphosphatidyl-ethanolamine); N-(7-nitrobenz-2-oxa-1,3-diazol-4-yl)-dipalmitoylphosphatidylethanolamine); and fluorescein-labeled wheat germ agglutinin.

In particular embodiments of the above-described methods, the second fluorescent dye is selected from the group consisting of: an oxonol that acts as the fluorescent acceptor; bis(1,3-dialkyl-2-thiobarbiturate)trimethineoxonols; bis(1,3-dihexyl-2-thiobarbiturate)trimethineoxonol; bis(1,3-dialkyl-2-thiobarbiturate) quatramethineoxonols; bis(1,3-dialkyl-2-thiobarbiturate)pentamethineoxonols; bis(1,3-dihexyl-2-thiobarbiturate)pentamethineoxonol; bis(1,3-dibutyl-2-thiobarbiturate)pentamethineoxonol); and bis(1,3-dialkyl-2-thiobarbiturate)hexamethineoxonols.

In a particular embodiment of the above-described methods, the cells are eukaryotic cells. In another embodiment, the cells are mammalian cells, preferably human cells. In other embodiments, the cells are L cells L-M(TK⁻) (ATCC CCL 1.3), L cells L-M (ATCC CCL 1.2), HEK 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26), or MRC-5 (ATCC CCL 171).

In assays to identify activators or inhibitors of cation channels containing human HCN3 protein, it may be advantageous to co-express another cation channel subunit besides human HCN3. In particular, it may be advantageous to co-express another HCN family member subunit (e.g., HCN1, HCN2, or HCN4). Preferably, this is done by co-transfecting into the cells an expression vector encoding the other HCN family member subunit.

The present invention also includes assays for the identification of inhibitors of cation channels containing human HCN3 protein that are based upon modulation of the growth phenotype of trk1Δtrk2Δ mutant yeast that also express cation channels containing human HCN3. The products of the yeast trk1 and trk2 genes are high affinity potassium transporters and their expression in wild type yeast allows growth under conditions in which the concentration of K⁺ in the medium is very low (e.g., <50 μM). Deletion, or inactivation, of these two genes abolishes high affinity K⁺ uptake and results in impaired growth in potassium limited (e.g, <7 mM) media. In addition, growth of trk1Δtrk2Δ yeast is also impaired by low (<3.0) pH even in the presence of otherwise permissive K⁺ concentrations (Nakamura & Gaber, 1999, Methods. Enz. 293:89-104). Heterologous expression of a human HCN3 cation channel in trk1Δtrk2Δ yeast could rescue the mutant growth phenotype. That is, expression of such a channel could restore wild type growth to these cells in limiting K⁺ or low pH. Thus, inhibitors of human HCN3 cation channels will negate its effect in these mutant yeast and result in their reversion to the mutant growth phenotype (i.e., impaired growth in low K⁺ or low pH). Thus, the present invention includes a method of identifying inhibitors of cation channels containing human HCN3 protein comprising:

(a) providing a yeast strain that has been engineered to

(1) have inactivated trk1 and trk2 genes and

(2) heterologously express a cation channel containing human HCN3 protein;

(b) exposing the yeast to a substance;

(c) measuring the growth rate of the yeast in the presence of the substance under either limiting K⁺ concentration or low pH and in the absence of the substance under either limiting K⁺ concentration or low pH;

(d) comparing the growth rates measured in step (c) in the presence and in the absence of the substance;

wherein if the growth rate in the presence of the substance is less than the growth rate in the absence of the substance then the substance is an inhibitor of cation channels containing human HCN3 protein.

In certain embodiments, the yeast trk1 and trk2 genes have been inactivated by deletion or mutagenesis.

Growth of the yeast is measured in media containing either 1) limiting K⁺ (e.g., <7 mM K⁺) or 2) permissive K⁺ and low pH (e.g., 100 mM K⁺ and pH <3.0). Growth rate may simply be measured as turbidity of the culture (e.g., as absorbance at 700 nm) as a function of time, or may be measured by other methods known in the art. Growth rate may also be measured in an all or none fashion by measuring the yeast's ability to form colonies in the presence of the absence of the substance.

While the above-described methods are explicitly directed to testing whether “a” substance is an activator or inhibitor of cation channels containing human HCN3 protein, it will be clear to one skilled in the art that such methods can be used to test collections of substances, e.g., combinatorial libraries, natural products extracts, to determine whether any members of such collections are activators or inhibitors of cation channels containing human HCN3 protein. Accordingly, the use of collections of substances, or individual members or subsets of such members of such collections, as the substance in the above-described methods is within the scope of the present invention.

The present invention includes pharmaceutical compositions comprising activators or inhibitors of cation channels comprising human HCN3 protein that have been identified by the herein-described methods. The activators or inhibitors are generally combined with pharmaceutically acceptable carriers to form pharmaceutical compositions. Examples of such carriers and methods of formulation of pharmaceutical compositions containing activators or inhibitors and carriers can be found in Gennaro, ed., Remington's Pharmaceutical Sciences, 18^{th} Edition, 1990, Mack Publishing Co., Easton, Pa. To form a pharmaceutically acceptable composition suitable for effective administration, such compositions will contain a therapeutically effective amount of the activators or inhibitors.

Therapeutic or prophylactic compositions are administered to an individual in amounts sufficient to treat or prevent conditions where the activity of cation channels containing human HCN3 protein is abnormal. The effective amount can vary according to a variety of factors such as the individual's condition, weight, gender, and age. Other factors include the mode of administration. The appropriate amount can be determined by a skilled physician. Generally, an effective amount will be from about 0.01 to about 1,000, preferably from about 0.1 to about 250, and even more preferably from about 1 to about 50 mg per adult human per day.

Compositions can be used alone at appropriate dosages. Alternatively, co-administration or sequential administration of other agents can be desirable.

The compositions can be administered in a wide variety of therapeutic dosage forms in conventional vehicles for administration. For example, the compositions can be administered in such oral dosage forms as tablets, capsules (each including timed release and sustained release formulations), pills, powders, granules, elixirs, tinctures, solutions, suspensions, syrups and emulsions, or by injection. Likewise, they can also be administered in intravenous (both bolus and infusion), intraperitoneal, subcutaneous, topical with or without occlusion, or intramuscular form, all using forms well known to those of ordinary skill in the pharmaceutical arts.

Compositions can be administered in a single daily dose, or the total daily dosage can be administered in divided doses of two, three, four or more times daily. Furthermore, compositions can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen.

The dosage regimen utilizing the compositions is selected in accordance with a variety of factors including type, species, age, weight, sex and medical condition of the patient; the severity of the condition to be treated; the route of administration; the renal, hepatic and cardiovascular function of the patient; and the particular composition thereof employed. A physician of ordinary skill can readily determine and prescribe the effective amount of the composition required to prevent, counter or arrest the progress of the condition. Optimal precision in achieving concentrations of composition within the range that yields efficacy without toxicity requires a regimen based on the kinetics of the composition's availability to target sites. This involves a consideration of the distribution, equilibrium, and elimination of a composition.

The inhibitors and activators of cation channels containing human HCN3 protein will be useful for treating a variety of diseases involving excessive or insufficient cation channel activity.

Expression of human HCN3 in the human brain, heart, pancreas and several other tissues was seen by Northern blot analysis. This suggests that inhibitors and activators of cation channels containing human HCN3 protein are likely to be useful for the treatment of neurodegenerative diseases, cognitive and sensory disorders, pain, cardiac brady- and tachy-arrhythmias, ataxias, fertility disorders, hepatic dysfunction, pancreatic disorders (including diabetes), heart failure, inflammatory disease, and diabetic neuropathy.

The human HCN3 nucleic acids and proteins of the present invention are useful in conjunction with screens designed to identify activators and inhibitors of other ion channels. When screening compounds in order to identify potential pharmaceuticals that specifically interact with a target ion channel, it is necessary to ensure that the compounds identified are as specific as possible for the target ion channel. To do this, it is necessary to screen the compounds against as wide an array as possible of ion channels that are similar to the target ion channel. Thus, in order to find compounds that are potential pharmaceuticals that interact with ion channel A, it is not enough to ensure that the compounds interact with ion channel A (the “plus target”) and produce the desired pharmacological effect through ion channel A. It is also necessary to determine that the compounds do not interact with ion channels B, C, D, etc. (the “minus targets”). The methods used to determine that a compound that is a drug candidate does not interact with minus targets are often referred to as “counterscreens.” In general, as part of a screening program, it is important to use as many minus targets in counterscreens as possible (see Hodgson, 1992, Bio/Technology 10:973-980, at 980). Human HCN3 protein, DNA encoding human HCN3 protein, and recombinant cells that have been engineered to express human HCN3 protein have utility in that can be used as “minus targets” in screening programs designed to identify compounds that specifically interact with other ion channels. For example, Wang et al., 1998, Science 282:1890-1893 have shown that KCNQ2 and KCNQ3 form a heteromeric potassium ion channel know as the “M-channel.” The M-channel is an important target for drug discovery since mutations in KCNQ2 and KCNQ3 are responsible for causing epilepsy (Biervert et al., 1998, Science 279:403-406; Singh et al., 1998, Nature Genet. 18:25-29; Schroeder et al., Nature 1998, 396:687-690). A screening program designed to identify activators or inhibitors of the M-channel would benefit greatly by the use of cation channels comprising human HCN3 protein as minus targets.

Accordingly, the present invention includes methods for identifying drug candidates that modulate ion channels where the methods encompass using human HCN3 in a counterscreen. Such methods comprise:

(a) determining that a compound is an activator or an inhibitor of an ion channel where the ion channel does not comprise human HCN3; and

(b) determining that the compound is not an activator or an inhibitor of ion channels comprising human HCN3.

Of course, human HCN3 may also be valuable in counterscreens where the primary drug target is not an ion channel. Thus, the present invention includes a method for determining that a drug candidate is not an activator or inhibitor of human HCN3 comprising:

(a) selecting a drug target that is not human HCN3;

(b) screening a collection of compounds to identify a compound that is an activator or an inhibitor of the drug target; and

(c) determining that the compound identified in step (b) is not an activator or an inhibitor of human HCN3.

The present invention also includes antibodies to the human HCN3 protein. Such antibodies may be polyclonal antibodies or monoclonal antibodies. The antibodies of the present invention can be raised against the entire human HCN3 protein or against suitable antigenic fragments that are coupled to suitable carriers, e.g., serum albumin or keyhole limpet hemocyanin, by methods well known in the art. Methods of identifying suitable antigenic fragments of a protein are known in the art. See, e.g., Hopp & Woods, 1981, Proc. Natl. Acad. Sci. USA 78:3824-3828; and Jameson & Wolf, 1988, CABIOS (Computer Applications in the Biosciences) 4:181-186.

For the production of polyclonal antibodies, human HCN3 protein antigenic fragments, coupled to a suitable carrier, are injected on a periodic basis into an appropriate non-human host animal such as, e.g., rabbits, sheep, goats, rats, mice. The animals are bled periodically and sera obtained are tested for the presence of antibodies to the injected human HCN3 protein or antigenic fragment. The injections can be intramuscular, intraperitoneal, subcutaneous, and the like, and can be accompanied with adjuvant.

For the production of monoclonal antibodies, human HCN3 protein or antigenic fragments, coupled to a suitable carrier, are injected into an appropriate non-human host animal as above for the production of polyclonal antibodies. In the case of monoclonal antibodies, the animal is generally a mouse. The animal's spleen cells are then immortalized, often by fusion with a myeloma cell, as described in Kohler & Milstein, 1975, Nature 256:495-497. For a fuller description of the production of monoclonal antibodies, see Antibodies: A Laboratory Manual, Harlow & Lane, eds., Cold Spring Harbor Laboratory Press, 1988.

Gene therapy may be used to introduce human HCN3 protein into the cells of target organs. Nucleotides encoding human HCN3 protein can be ligated into viral vectors, which mediate transfer of the nucleotides by infection of recipient cells. Suitable viral vectors include retrovirus, adenovirus, adeno-associated virus, herpes virus, vaccinia virus, lentivirus, and polio virus based vectors. Alternatively, nucleotides encoding human HCN3 protein can be transferred into cells for gene therapy by non-viral techniques including receptor-mediated targeted transfer using ligand-nucleotide conjugates, lipofection, membrane fusion, or direct microinjection. These procedures and variations thereof are suitable for ex vivo as well as in vivo gene therapy. Gene therapy with wild type human HCN3 proteins will be particularly useful for the treatment of diseases where it is beneficial to elevate cation channel activity. Gene therapy with a dominant negative mutant of human HCN3 protein will be particularly useful for the treatment of diseases where it is beneficial to decrease cation channel activity.

The following non-limiting examples are presented to better illustrate the invention.

### EXAMPLE 1

### Identification and Cloning of Human HCN3 cDNA

Full length HCN3 was cloned as two separate overlapping fragments by PCR: 1) the amino terminus from EST AI571225 to a region downstream (3′) to the cyclic nucleotide binding domain and 2) a region 5′ to S4 to the stop codon were designed from the sequence obtained from the genomic DNA database searches. Two identical cDNAs, encoding the amino terminal sequence, were obtained by standard PCR techniques using the following primer pair:

5′ TGGGCGCCATGGAGGCAGAGCAGCGGCCG 3′ (SEQ.ID.NO.:15)

5′ TTTCCACTAAGCTGAGCTCCT 3′ (SEQ.ID.NO.:16)

The preS4 to stop cDNAs was cloned by PCR with the following primer pair:

5′ GAGCCACGGTTGGACGCTGAG 3′ (SEQ.ID.NO.:17)

5′ AAAGGTTTTACATGTTGGCAGAAAGCTG 3′ (SEQ.ID.NO.:18)

Three clones were sequenced. When all five PCR sequences were aligned and compared to the corresponding genomic sequence, two polymorphisms were found. One nucleotide polymorphism was at position 1051 and one was at position 1795. The cDNA encoding the full length ORF was then constructed from the two partial cDNAs by standard molecular biologic techniques.

### EXAMPLE 2

### Analysis of the Expression of Human HCN3

Northern blots of poly(A⁺) RNA isolated from human heart, brain, placenta, lung, liver, skeletal muscle, kidney, pancreas, spleen, thymus, prostate, testis, uterus, small intestine, colon, and peripheral blood leukocytes were purchased from Clontech (Palto Alto, Calif.) and probed with a ³²P-labeled oligonucleotide probe derived from EST AI571225. The sequence of the sense stand of the probe is:

5′ CGAAGGGGCGACCCCTGGACTGGAGGCGGTGCCTCCCGTTGCTCCCCCG CCTGCGACCG 3′ (SEQ.ID.NO.:19)

The probe was constructed from two overlapping synthetic oligonucleotides that were annealed and filled in in the prescence of all four [³²P]dNTPs and the Klenow fragment of DNA polymerase. The hybridization was carried out in 5× SSPE, 2× Denhardt's solution, 0.5% SDS, 100 μg/ml salmon sperm DNA at 42° C. overnight. Blots were washed stepwise in 2×SSC, 0.05% SDS at 42° C. 3 times for 15-20 minutes, followed by 1×SSC, 0.05% SDS at 50° C. 3 times for 15-20 minutes. Hybridization was detected by exposure of the blots to Kodak CAR X-ray film.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Various publications are cited herein, the disclosures of which are incorporated by reference in their entireties.

What is claimed is:

1. An isolated DNA comprising nucleotides encoding human HCN3.

2. The DNA of claim 1 comprising nucleotides encoding a polypeptide having an amino acid sequence selected from the group consisting of SEQ.ID.NOs.:2, 4, and 6.

3. The DNA of claim 1 comprising a nucleotide sequence selected from the group consisting of: SEQ.ID.NO.:1, SEQ.ID.NO.:3, SEQ.ID.NO.:5, positions 9 to 2330 of SEQ.ID.NO.:1, positions 9 to 2330 of SEQ.ID.NO.:3, and positions 9 to 2330 of SEQ.ID.NO.:7.

4. An isolated DNA that hybridizes under stringent conditions to the DNA of claim 3 and that encodes a protein having substantially the same biological activity as human HCN3.

5. An expression vector comprising the DNA of claim 3.

6. A recombinant host cell comprising the DNA of claim 3.

7. DNA, substantially free of other nucleic acids, comprising nucleotides encoding a polypeptide having an amino acid sequence selected from the group consisting of SEQ.ID.NOs.:2, 4, and 6.

8. An isolated human HCN3 protein.

9. The protein of claim 7 comprising an amino acid sequence selected from the group consisting of SEQ.ID.NOs.:2, 4, and 6.

10. The protein of claim 8 containing a single amino acid substitution.

11. The protein of claim 8 containing two or more amino acid substitutions where the amino acid substitutions do not occur in conserved positions.

12. A protein, substantially free of other proteins, comprising an amino acid sequence selected from the group consisting of SEQ.ID.NOs.:2, 4, and 6.

13. An antibody that binds specifically to a human HCN3 protein.

14. A DNA or RNA oligonucleotide probe comprising at least 10 contiguous nucleotides from SEQ.ID.NO.:1, 3, or 5.

15. A method of identifying substances that bind to cation channels containing human HCN3 protein comprising:
(a) providing cells expressing a cation channel containing human HCN3 protein;
(b) exposing the cells to a substance that is not known to bind cation channels containing human HCN3 protein;
(c) determining the amount of binding of the substance to the cells;
(d) comparing the amount of binding in step (c) to the amount of binding of the substance to control cells where the control cells are substantially identical to the cells of step (a) except that the control cells do not express human HCN3 protein;
where if the amount of binding in step (c) is greater than the amount of binding of the substance to control cells, then the substance binds to cation channels containing human HCN3 protein.

16. A method of identifying substances that bind cation channels containing human HCN3 protein comprising:
(a) providing cells expressing cation channels containing human HCN3 protein;
(b) exposing the cells to a compound that is known to bind to the cation channels containing human HCN3 protein in the presence and in the absence of a substance not known to bind to cation channels containing human HCN3 protein;
(c) determining the amount of binding of the compound to the cells in the presence and in the absence of the substance;
where if the amount of binding of the compound in the presence of the substance differs from that in the absence of the substance, then the substance binds cation channels containing human HCN3 protein.

17. A method of identifying activators or inhibitors of cation channels containing human HCN3 protein comprising:
(a) recombinantly expressing human HCN3 protein in a host cell so that the recombinantly expressed human HCN3 protein forms cation channels either by itself or by forming heteromers with other cation channel subunit proteins;
(b) measuring the biological activity of the cation channels formed in step (a) in the presence and in the absence of a substance not known to be an activator or an inhibitor of cation channels containing human HCN3 protein;
where a change in the biological activity of the cation channels formed in step (a) in the presence as compared to the absence of the substance indicates that the substance is an activator or an inhibitor of cation channels containing human HCN3 protein.
